(19) United States
(12) Patent Application Publication
(10) Pub. No.: US 2023/0263153 A1
(43) Pub. Date: Aug. 24, 2023

(54) APPARATUS AND METHOD FOR SUPPRESSING SCENT ORIGINATING IN ENCLOSED STRUCTURE

(71) Applicant: Manifold Investments, LLC, Jonesville, MI (US)

(72) Inventor: Scott Manifold, Jonesville, MI (US)

(21) Appl. No.: 18/060,885

(22) Filed: Dec. 1, 2022

Related U.S. Application Data
(60) Provisional application No. 63/313,049, filed on Feb. 23, 2022.

(51) Int. Cl. A01M 31/02; A61L 2/20; A61L 9/03

(52) U.S. Cl. CPC A01M 31/02; A61L 2/202; A61L 9/032; A61L 9/035

(57) ABSTRACT

An apparatus and method for reducing scent emanating from a substantially enclosed structure such as a hunting blind. A fan in a fluid conduit draws odor-laden air from the internal environment and passes it through an ozone-rich vicinity provided for example by one or more ozone generators, then exhausts the treated deodorized air to the external environment. Fresh air is introduced into the negative-pressure enclosure from the external environment through one or more gaps or cervices in the structure.

### CROSS REFERENCE TO RELATED APPLICATIONS

This application claims priority to U.S. provisional application 63/313,049 filed Feb. 23, 2022, which is fully and expressly incorporated herein by reference.

### BACKGROUND OF THE INVENTION

Hunters, wildlife enthusiasts and wildlife photographers all contend with the control of human scent and other odors associated with themselves or their activities afield. Animals have highly effective olfactory systems that allow them to detect foreign odors and thereby associate them with danger. This makes approaching them much more difficult.

There are systems that claim to be effective using ozone - a strong oxidizing agent - to eradicate or substantially suppress odors deemed alarming to wildlife or to emit a less offensive bleach-like odor, thereby theoretically not alarming animals. One example is U.S. 8,663,553B2 (“Elrod”), the entirety of which is expressly incorporated herein by reference. The inherent problem with the system and method of Elrod and similar approaches to suppressing scent is that it requires that a human place himself in an environment such as a hunting blind or other enclosure that 1) has ozone levels that are so high as to be unsafe or 2) so low as to be ineffective at controlling human-related odors and scent.

It is difficult or impossible to control the generation and position of ozone at acceptable efficacy and risk levels. As noted in Elrod, OSHA has released acceptable time-weighted exposure levels, for example limiting permissible exposure limit (PEL) as an eight-hour time-weighted average value of 0.1 ppm ozone in air or a two-hour time-weighted average value of 0.2 ppm ozone in air. The OSHA short term exposure limit (STEL) is 0.3 ppm over a 15-minute period, not to be repeated more than two times in an eight-hour period. According to the EPA, concentrations of ozone considerably higher than these standards are possible when a user uses an indoor ozone generator, even when following the manufacturer’s operating instructions. The U.S. Environmental Protection Agency has summarized a number of scientific studies on its website, accessible at www.epa.gov/indoor-air-quality-iaq/ozone-generators-are-sold-air-cleaners. These exposures are easily exceeded such as when the proper ratio of fresh air to ozone thru ventilation is not achieved. Ozone generators that are sold as air cleaners can give rise to side effects including decreased lung function, aggravation of asthma, throat irritation, chest pain and shortness of breath, inflammation of lung tissue, and higher susceptibility to respiratory infection. Since every enclosure placed in the wild will vary dramatically in its air-tightness, one would need to carry a device to measure the exposure levels or wait to feel the side effects of ozone poisoning.

If one were theoretically able to control safely the concentration of ozone in a structure, efficacy is doubtful. The same EPA website states that an ozone generator, if operating at concentrations that do not exceed public health standards, has little potential to remove indoor air contaminants, is ineffective at removing many odor-causing chemicals, and does not effectively remove viruses, bacteria, mold, or other biological pollutants.

Current solutions to control scent in enclosed or semi-enclosed structures, such as hunting blinds, may include generation of one or more “curtains” of ozone around the structure, such as disclosed in Elrod, or immersing the occupants in an ozone-rich environment. Both approaches are difficult to control and can be hazardous to health. In either approach, the ozone levels must be sufficiently high to have the scents controlled or eliminated. In the curtain technique, effective scent control typically requires ascertaining wind direction and speed to eliminate or reduce scent produced within the structure from blowing in the general direction of the observation area. However, any change in wind speed, slight shift in wind direction, or internal ventilation parameters may result in dramatic reduction in efficacy or exposure to unhealthy levels of ozone. Ozone generated within a structure to mask human and other scents at the source may be less subject to variables of wind, but carry a risk of harmful side effects when operated at sufficiently high levels to achieve acceptable efficacy.

Reducing scent transmission by restricting substantially all odors emanating from an enclosure is both difficult and problematic. OSHA standards regarding CO₂ (carbon dioxide) levels provide a useful comparison to the dangers of ozone and the importance of maintaining adequate ventilation. OSHA warns that excessive CO₂ can cause headache, sweating, rapid breathing, increased heartbeat, shortness of breath, dizziness, mental depression, visual disturbances, or shaking. These symptoms are commonly referred to as hypercarbia. If a person attempts to place himself in relatively air-tight enclosure for the said purpose of avoiding detection by scent, CO₂ levels begin to build immediately from normal breathing (biological respiration). Within one or two hours CO₂ may exceed 5000 parts per million. The inventor has conducted tests in which two adult occupants in a fairly air tight hunting enclosure of (6′ x 6′ x 6′6″ tall) registered CO₂ levels of 6000 ppm after 30 minutes, 7750 ppm after 60 minutes, and 8070 ppm at the 65 minute mark after which tests were terminated for safety concerns. A similar test performed with three adult occupants found 9000 ppm in 90 minutes before terminating test for safety. Of course potentially dangerous levels will occur faster or slower depending on the size and air tightness of the enclosure and the number of people in the enclosure. OSHA has established permissible exposure limits (PEL) for CO₂ of 5000 ppm averaged over an 8-hour workday. These dangers would of course be exacerbated if ozone were simultaneously being generated within the air-tight enclosure, presumably resulting in comparable concentrations of ozone gas and potentially raising safety concerns even more serious than CO₂.

What is needed is an apparatus and method to effectively control human scent and odor emanating from a substantially enclosed area such as a hunting blind without threatening the health and safety of occupants.

### BRIEF SUMMARY OF THE INVENTION

Disclosed is an apparatus and method for reducing scent emanating from a substantially enclosed structure’s internal environment to the external environment. The structure such as a hunting blind or structure for observing wildlife, may be permanent, semi-permanent, or temporary. In one embodiment, the apparatus comprises a fluid conduit between the internal environment and the external environment, an ozone source within the fluid conduit, and a fan within the fluid conduit configured to draw scent-laden air from the internal environment through an inlet then to pass it across an ozone-rich zone then exhausting the treated air to the external environment at an exhaust outlet. The ozone may be generated by an electric or electronic ozone generator powered by an energy source such as a battery. The ozone-rich environment oxidizes and substantially neutralizes odors, such as human scent, as the interior air is passed thru the ozone-rich zone before being exhausted outside the structure in the form of de-scented air, or in some embodiments exhausts air having a mildly pungent, bleach-like smell known to be associated with ozone generating devices. Fresh air is introduced into the negative-pressure enclosure from the outside through one or more gaps or cervices resulting from imperfect fit of walls, ceiling, etc., or in some embodiments through vents or windows. If a person is in a very air tight enclosure in an attempt to control scent, the invention allows the occupant(s) to moderately open a vent or window while maintaining some negative pressure with the structure (as provided by the flow of air inwardly through said open vent or ajar window) and introduce fresh air to the occupants, lowering levels of CO₂, and effectively deodorizing air exhausted from the enclosure.

### BRIEF DESCRIPTION OF THE SEVERAL VIEWS OF THE DRAWINGS

These and other features and advantages of the present invention will become more readily appreciated when considered in connection with the following detailed description and appended drawings, wherein:

FIG. 1 is a cross section of a substantially enclosed structure showing the apparatus mounted on a sidewall and the airflow while the apparatus is operating.

FIG. 2 shows a cross section of a housing comprising an inlet port and an outlet port, and enclosing a pair of ozone generators and a fan near the outlet port.

FIG. 3 shows a cross section of an embodiment of the apparatus having a housing enclosing a pair of ozone generators and a fan, a pair of retainers configured to secure the housing to a wall of the structure, and section of flexible duct.

FIG. 4 is a cross section of a substantially enclosed soft-sided structure showing another embodiment of the apparatus showing the housing suspended from the ceiling and flexible conduit attached to the housing at both the inlet port and outlet port.

FIG. 5 is a cut-away view of another embodiment of the apparatus wherein the housing is comprised of PVC pipe and the fan is near the inlet port.

### DETAILED DESCRIPTION OF THE INVENTION

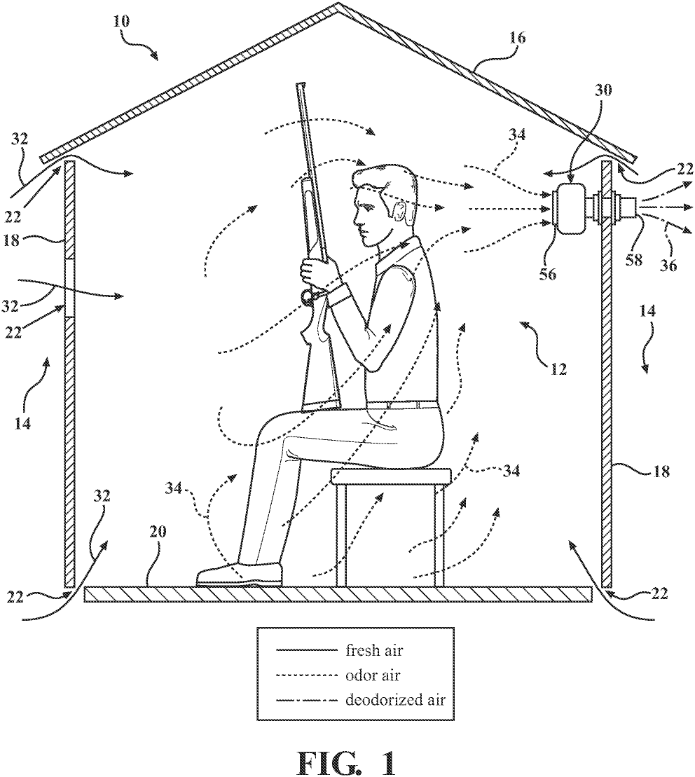

Referring to the figures, wherein like numerals indicate like or corresponding parts throughout the several views, FIG. 1 depicts a structure 10 having a human occupant. The occupant has odor, or scent, that originates from the occupant himself (e.g. clothing, sweat, soap, etc.) or from items present in the enclosure derived from human activity that wildlife may associate with humans and/or danger (e.g. food, fuel, ammunition, etc.). For purposes of the claims and specification, odor and scent are synonymous. FIG. 1 depicts structure 10 as having a rigid roof 16 and rigid sidewalls 18 that together define an enclosed internal environment 12 and an external environment 14. Usually, but not necessarily, external environment 14 is outdoors; however, enclosed structure 10 could be located within a much larger structure, such as within an enclosure at a zoo. The internal environment 12 comprises atmospheric scent including human scent. Structure 10 may optionally have a floor, or may stand directly on the earth. Roof 16 and sidewalls 18 may be comprised of any material that wholly or substantially restricts air, such as plywood, metal, glass, fiberglass or plastic boards or panels. In some embodiments, structure 10 is comprised of a fabric, such as nylon or canvas, that similarly restricts air flow but that may not be impermeable to scented airflow, with the fabric typically supported by a framework such as tubes, poles, and/or rods.

Structure 10 is not wholly impermeable to odor located within the internal environment 12 at least because some air circulation is necessary to provide fresh air for the occupant to breathe and carbon dioxide to escape. Fresh air 32 enters structure 10 through gaps 22 or cervices in structure 10, and/or through vents or windows fabricated in structure 10. Such gaps may arise from imperfect fit of between adjacent sidewalls 18, imperfect fit or between a sidewall 18 and the earth (or floor) or roof 16, or through vents or windows built as features into one or more areas of structure 10. FIG. 1 shows gaps 22 at various locations, including a vent or window shown on the left side of structure 10 through which the occupant can see external environment 14. If there is no means of deodorizing air within the structure, odor air 34 would emanate from internal environment 12 to external environment 14 through these same gaps, and quite possibly also through roof 16 and sidewalls 18 if they are not perfectly impermeable (such as a structure comprised of nylon fabric).

FIG. 1 shows descenting apparatus 30, in this embodiment mounted on a sidewall 18. As described in greater detail below, descenting apparatus 30 comprises a fluid conduit between internal environment 12 and external environment 14, commencing at inlet 56 and terminating at outlet 58. Descenting apparatus 30 also comprises a means of creating negative pressure in the internal environment 12 relative to external environment 14, such as an electric fan. In operation, de-scenting apparatus 30 draws odor air 34 air from the internal environment 12 into the fluid conduit at inlet 56 then exhausts the deodorized air 36 outside of structure 10 through outlet 58 after the air passes through one or more zones located within the fluid conduit in which a descenting agent is present. The descenting agent may comprise ozone, an ionized gas including that produced by an ion generator or a corona discharge generator or a hydroxyl generator, photocatalytic oxidation, and/or a gaseous compound that masks scent, such as a commercial scent eliminator, e.g. Nose Jammer® field spray, or a combination of one or more of the above. The descenting agent may be comprised of gaseous forms such as ozone or ionized gas, and/or an aerosolized liquid, or vaporized solid. In a preferred embodiment the descenting agent comprises ozone.

The resulting negative pressure within internal environment 12 causes fresh air 32 to enter structure 10 through one or more gaps 22. In the embodiment shown in FIG. 1, the fluid conduit begins where odor air 34 enters descenting apparatus 30 at inlet 56 and the fluid conduit terminates where deodorized air 36 leaves descenting apparatus 30 at outlet 58. In the embodiment shown in FIG. 1, much of the fluid conduit is within housing 44, with a relatively short section of the fluid conduit extending from housing 44 and through sidewall 18 allowing deodorized air 36 to be exhausted to the external environment 14 at outlet 58.

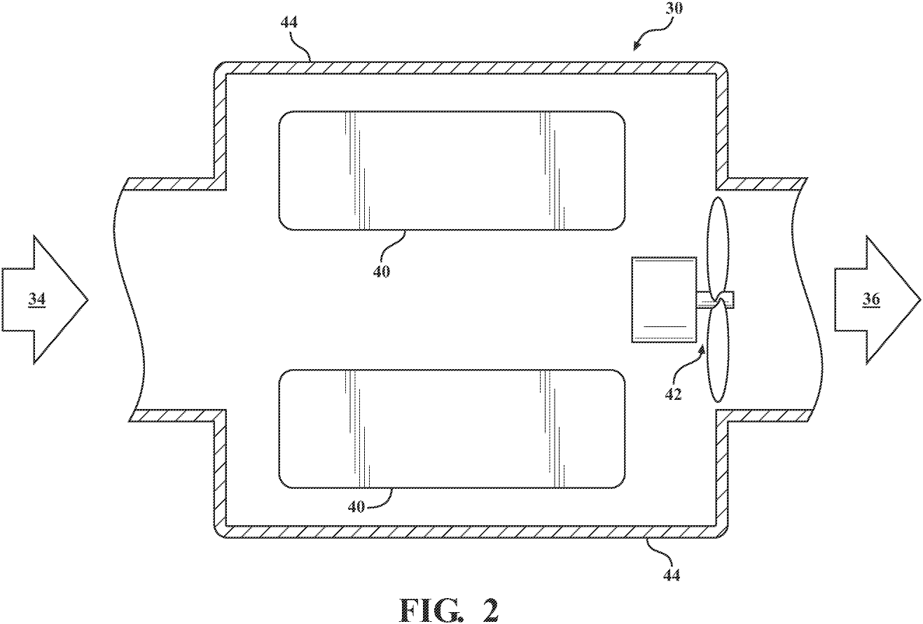

FIG. 2 shows a cross section of an embodiment of descenting apparatus 30 structurally consistent with the embodiment shown in FIG. 1. In this embodiment the interior of housing 44 is part of the fluid conduit between internal environment 12 and external environment 14. Housing 44 may be any configuration comprised of any substantially impermeable material such as metal, plastic, glass, fiberglass, polycarbonate, or carbon fiber, may be rigid or semi-rigid, and may enclose all or fewer than all components of de-scenting apparatus 30.

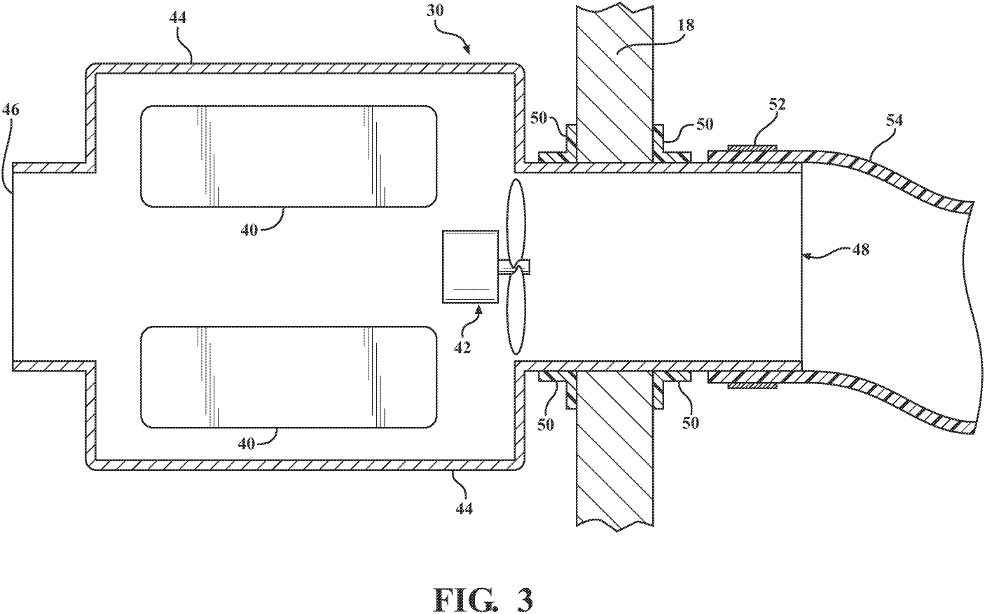

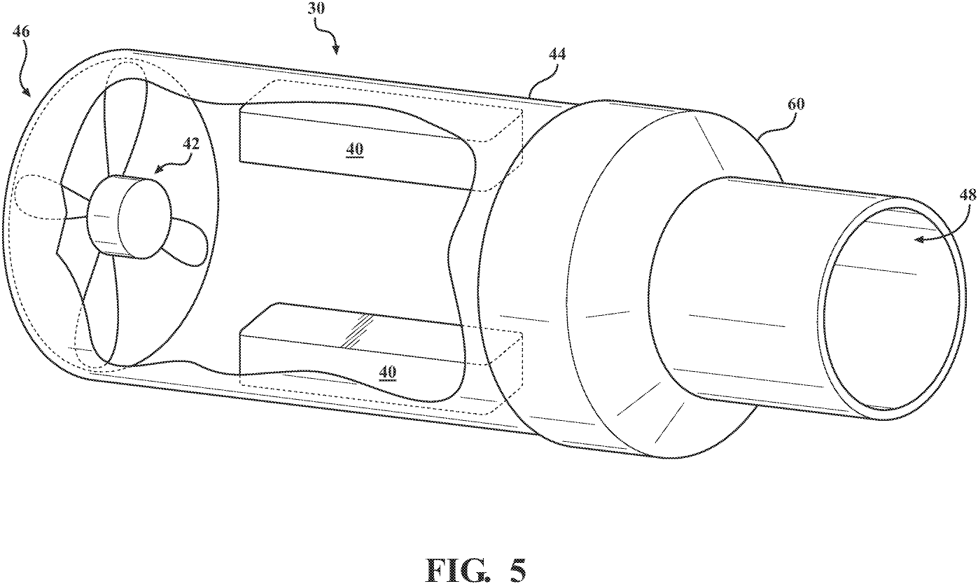

Housing 44 comprises an inlet port 46 which receives odor air 34. Inlet port 46 is fluidly connected to outlet port 48. An ozone-rich zone is created within housing 44 by one or more ozone generators 40. Odor air 34 drawn from internal environment 12 through inlet port 46 passes through the ozone-rich zone after which it becomes deodorized air 36, which exits through outlet port 48. Air is drawn through the fluid conduit by at least one fan 42. In the embodiments shown in FIGS. 2 and 3, fan 42 is situated within housing 44 near outlet port 48, but fan 42 may be located anywhere within the fluid conduit, including near the inlet port as shown in FIG. 5 or even beyond the confines of housing 44. Fan 42 may be of any type, e.g., axial, vaned, centrifugal, or “bladeless.” In one embodiment, fan 42 is powered by an electrical source (not shown), such as a DC battery, and having variable speed controller (not shown) that a user may adjust to create appropriate negative pressure in internal environment 12 relative to external environment 14 to assure satisfactory deodorizing of odor air 34 drawn from internal environment 12 without creating unnecessary and/or uncomfortable draftiness in structure 10 from incoming fresh air 32. In some embodiments, housing 44 may comprise acoustical dampening features, such as acoustic foam, to minimize fan noise.

In the embodiments shown in the figures, one or more ozone generators 40 are located within the fluid conduit, and more specifically within housing 44. Ozone generators operate by providing an area of high electric potential, causing oxygen molecules to break apart and reform as O₃. Ozone generators are commercially available in many sizes and may be configured as an integrated unit or separated into components, e.g., 1) a voltage and/or amperage controller connected to 2) an electric supply such as a DC battery and 3) one or more surfaces configured to provide an electric field, usually ceramic plates, sometimes tubes, although other configurations are possible. These components, well known to those skilled in the art of ozone generators, are not individually shown in the drawings. Instead, ozone generator 40 is schematically represented as a simple rectangular “box” representing the source of generated ozone, such as a pair of energized ceramic plates. Similar to fan 42, output from ozone generator 40 may be varied by, e,g,, adjusting the voltage or amperage of electricity provided to the ceramic plate or tube. The electrical source and electrical controller powering the ceramic plates (or tubes, etc.) need not be located near the plates/tubes or within housing 44. In some embodiments, it may be preferable to power the ozone generator with a remotely located battery such as a 12V car battery, controlled by an electrical controller that may likewise be separate or near the electric power source. In other embodiments, two or more of the various ozone generator components might be co-located in, on, or near housing 44. Fan 42 may be sized differently to adequately remove odor air 34 from smaller or larger enclosures, or to accommodate the size and/or number of ozone generators 40 depending on the mg of ozone generated. Descenting apparatus may further comprise one or more selector switches to select differing speeds on the fan or control ozone output. Housing 44 may comprise hinges and/or one or more latches (not shown) to facilitate access to the interior of the housing 44 for cleaning or replacement of fan 42 or ozone generator 40 or other maintenance.

More broadly, it will be appreciated that ozone generator 40 and fan 42 may be located anywhere in the fluid conduit between inlet 56 and outlet 58, in the same proximity or remote from one another, and/or configured with fan 42 either upstream or downstream of ozone generator 40, with similar functional results because they are fluidly connected. Thus in some embodiments (not shown) descenting apparatus 30 may have a fluid conduit with a fan and one or more ozone generators located therein, but lack a separate housing 44. For purposes of the claims and specification, “fluid conduit” includes embodiments where fan 42 and/or ozone generator 40 are in fluid communication with the body of air moving through the primary fluid conduit but are not necessarily located within the primary fluid conduit itself, such as introducing ozone into an airstream through a tube, venturi device, or other inlet fluidly connected to the ozone source and to the primary fluid conduit.

Housing 44 may be configured for mounting on sidewall 18 using one or more mounting techniques of any variety well known to those skilled in the art, depending on the size, material, and configuration of the fluid conduit where it passes through the sidewall 18. One or more areas of housing 44 may be configured for mounting to one or both sides of sidewall 18. FIG. 3 shows a cross section of an embodiment of descenting apparatus 30 with housing 44 having an extended outlet port 48 extending through sidewall 18 and retained by one or more straps or flanges 50 encircling the outlet port 48. In other embodiments, the mounting configuration may essentially consist of a hole bored in sidewall 18 sized to ensure an appropriately snug fit of a portion of the fluid conduit, such as to receive a portion of outlet port 48, in which case the hole in the sidewall is itself the retainer where it encircles the fluid conduit. In some embodiments, the fluid conduit may comprise a combination of rigid portions, such as housing 44, and flexible portions, such as a rubber connector 54 attached to outlet port 48. Rubber connector 54 may be secured to outlet port 48 with a clamp 52, such as an appropriately sized standard hose clamp.

Some users may desire to install descenting apparatus 30 temporarily, so that it can be removed from a first structure 10 and transported to storage or installed in another structure or hunting blind. In some embodiments, descenting apparatus 30 is configured for convenient attachment and detachment by an occupant. For purposes of the claims and specification, “convenient” means attachment and detachment without tools or with only simple tools such as hand pliers, a screwdriver, and/or tools found on a common multipurpose pocket tool such as a Leatherman® multi-tool. Clamp 52 may, for example, comprise a thumb screw for more convenient attachment and detachment. Other means of convenient attachment/detachment are well known in the art, such as friction fit, impermanent adhesive, fitted collars, springs, tabs, detents, and numerous other well-known attachment mechanisms for joining conduit such as used in plumbing and HVAC applications. The combination and configuration of clamps, flexible vs. rigid fittings, and other components may vary with based on the characteristics of structure 10, the configuration of descenting apparatus 30, the size and shape of the internal environment 12, the conditions in the external environment 14, the desired location of inlet 56 and outlet 58, the degree of portability desired, and individual preference.

In another embodiment (not shown), instead of flange 50 the fluid conduit can be secured at sidewall 18 by a retainer comprising one or more components that complement the dimensions of the fluid conduit at the sidewall 18. For example, a fitting might be installed in or on sidewall 18 complementing the dimensions of outlet port 48, e.g., a female fitting sized to receive male outlet port 48. The juncture can be permanently or temporarily secured by simple friction fit, impermanent adhesive, or adhesive tape such as duct tape. In some embodiments, outlet port 48 (or whatever portion of the fluid conduit downstream of outlet port 48 that will exit through sidewall 18) is inserted into a complementary fitting that is more or less permanently installed in sidewall 18. Similar complementary fittings could be installed in numerous structures, thus facilitating quick removal and re-installation if the occupant moves between hunting blinds. The assembly includes A configured to [purpose of A]. In one or more embodiments, A can be defined by any one of B, C, or D. In one or more other embodiments, A can be defined by any combination of B, C, and D. In one or more other embodiments, the assembly can include one or more other components in addition to A that impart the same functionality as one or more of B, C, or D but to a lesser extent.

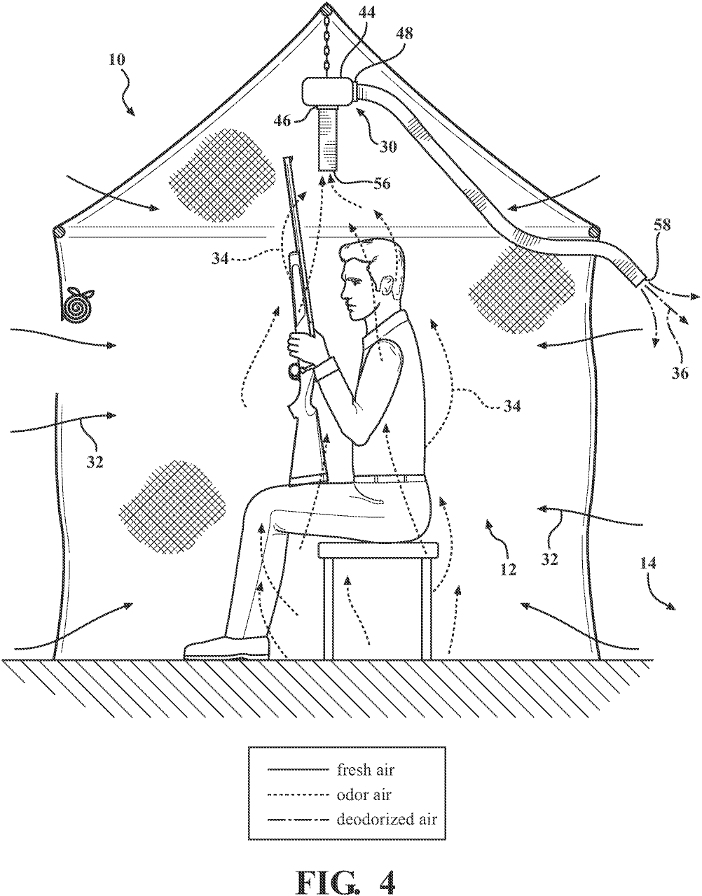

The fluid conduit may comprise any material suitable for conveying air such as rigid metal ducting, PVC pipe, or flexible ducting such as plastic duct reinforced with helical wire windings comparable to ducting used to exhaust moist air from a household clothes dryer. FIG. 4 shows an embodiment where structure 10 is soft-sided, i.e. comprised of a fabric material, such as nylon or rayon. A portion of the descenting apparatus 30, such as housing 44, may be suspended from an upper portion of structure 10. In other embodiments some or all of the apparatus may rest on the ground/floor, on a table, or be supported by a sidewall 18. The fluid conduit may comprise flexible ducting attached to inlet port 46 and another piece of flexible ducting attached to outlet port 48. The flexible ducting allows inlet 56 to be conveniently placed at a favorable location anywhere in internal environment 12, and the outlet 58 to be conveniently placed at a desired location outside structure 10 to exhaust deodorized air 36 to external environment 14 at a desired location, for example through a convenient vent or gap in the structure, e.g. between a sidewall 18 and the ground. Care should be taken to locate outlet 58 to minimize the amount of ozone-treated air 36 being immediately being drawn back into internal environment 12. Depending on the permeability of the fabric and construction features of structure 10, fresh air 32 may enter through the roof 16, sidewalls 18, and/or vents, windows, or other gaps.

The fluid conduit may comprise tubing or piping made of CPVC, PVC, ABS, Acrylic or other manufactured resins, compounds or materials, and may comprise flexible hose or tubing. In some embodiments housing 44 comprises an inlet port 46 and/or an outlet port 48 having dimensions compatible with standard dimension schedule 30 or schedule 40 PVC pipe or ABS pipe (e.g. 2-, 3-, 4- or 6-inch diameters) to facilitate attachment to other portions of the fluid conduit. In an exemplary embodiment shown in FIG. 5, housing 44 itself is comprised of rigid pipe material that may have standard PVC dimensions. As in other embodiments described above, housing 44 encloses one or more ozone generators 40 and fan 42. In this embodiment, fan 42 is installed near the inlet port 46 upstream of ozone generators 40. Standardized dimensions may facilitate durable and economical construction and attachment to other portions of the fluid conduit, such as a reducer 60 to facilitate connecting housing 44 to a different-size pipe. As before, not all electrical and related components of fan 42 and ozone generator 40 are shown in the figures; some components such as output controllers, voltage condensers, batteries, etc. may be electrically connected but remotely located from housing 44. Of course, housing 44 may incorporate suitable electrical connectors such as mated plugs, pins, etc. to conveniently connect controllers, a power source, and other components associated with fan 42 and ozone generator 40.

The descenting apparatus may comprise one or more barriers proximate to outlet 58 to prevent birds, insects, leaves, dirt, debris, etc. from entering the fluid conduit through outlet 58, such as a screen or louvres. One or more similar barriers may be located at proximate to inlet 56 and/or at inlet port 46 and/or outlet port 48, for example as a finger guard to prevent damage or injury caused by touching the fan 42 or electrical components while in operation.

When properly configured, descenting apparatus 30 reduces or virtually eliminates scent emanating from a substantially enclosed structure’s internal environment 12 to the external environment 14 by drawing odor air 34 into a fluid conduit having an inlet 56 located inside the structure 10 and exhausting ozone-treated air through an outlet 58 located outside the structure. Ozone is provided by an ozone source such as an ozone generator 40 at one or more vicinities within the fluid conduit. A fan 42 fluidly connected to the fluid conduit produces lower air pressure at inlet 56 relative to air pressure at outlet 58. Odor air 34 from the internal environment 12 becomes deodorized air 36 as it moves through the one or more ozone-rich vicinities in the fluid conduit. The deodorized air 36 is exhausted to external environment 14 at outlet 58. Occupants of structure 10 experience little or no exposure to the harmful effects of ozone. The exhausted deodorized air 36 is substantially de-scented, i.e. scents that may have subsisted in the enclosure exit the de-scenting apparatus with only an ozone or bleach type odor, which has little or no negative impact on the wildlife in the area outside the structure 10.

The invention of forced evacuation of odors within the structure through an ozone-rich vicinity is unlike any known system or method for using ozone to control or eliminate scent within an enclosure. This method renders scent control safe as there is no exposure to harmful effects of ozone to humans all while reliably defeating the wildlife’s ability to detect human odor or odors associated with human enterprises in the wild.

The foregoing invention has been described in accordance with the relevant legal standards, thus the description is exemplary rather than limiting in nature. Variations and modifications to the disclosed embodiment may become apparent to those skilled in the art and fall within the scope of the invention. Furthermore, particular features of one embodiment can replace corresponding features in another embodiment or can supplement other embodiments unless otherwise indicated by the drawings or this specification.

I claim:

1. An apparatus for reducing scent emanating from a substantially enclosed structure’s internal environment to the external environment, the apparatus comprising.

2. A fluid conduit between the internal environment and the external environment.

3. A descenting agent source within the fluid conduit; and.

4. A fan within the fluid conduit configured to exhaust air from the internal environment to the external environment.

5. The apparatus of claim 1, wherein the descenting agent comprises ozone.

6. The apparatus of claim 2 wherein the ozone is provided by an ozone generator.

7. The apparatus of claim 3 wherein the ozone generator is located within a housing, said housing comprising an inlet port fluidly connected to the internal environment and an outlet port fluidly connected to the external environment.

8. The apparatus of claim 4, wherein the fan is located within the housing.

9. The apparatus of claim 4, wherein the fan is located proximate to the inlet port.

10. The apparatus of claim 4, wherein the fan is located proximate to the outlet port.

11. The apparatus of claim 4, wherein the housing is configured to be mounted on a sidewall of the substantially enclosed structure.

12. The apparatus of claim 8, wherein the mounting configuration comprises a retainer encircling the fluid conduit proximate the outlet port.

13. The apparatus of claim 1, wherein the fluid conduit comprises PVC pipe.

14. The apparatus of claim 1, wherein the fluid conduit comprises flexible ducting.

15. The apparatus of claim 4, wherein the housing is configured to be suspended within the substantially enclosed structure.

16. The apparatus of claim 3, wherein the fan and the ozone generator are electrically connected to one or more power sources.

17. The apparatus of claim 13, wherein the rate of ozone generation is variable.

18. A method of reducing scent emanating from a substantially enclosed structure’s internal environment to the external environment, the method comprising.

19. Providing a fluid conduit having an inlet located inside the structure and an exhaust outlet outside the structure.

20. Providing a descenting agent at one or more vicinities within the fluid conduit.

21. Producing lower air pressure at the inlet relative to air pressure at the outlet.

22. Deodorizing air from the internal environment as it moves through the one or more vicinities in the fluid conduit; and.

23. Exhausting deodorized air to the external environment.

24. The method of claim 15, wherein the descenting agent comprises ozone.

25. The method of claim 16, wherein the ozone is provided by one or more ozone generators.

26. The method of claim 17, wherein the air pressure differential is produced by a fan located within the fluid conduit.

27. The method of claim 18, wherein the fluid conduit comprises a housing that encloses the fan and at least one ozone generator.

28. An apparatus for reducing scent emanating from a substantially enclosed structure’s internal environment to the external environment, the apparatus comprising.

29. A fluid conduit having an inlet inside the structure and an exhaust outlet outside the structure.

30. A housing comprising an inlet port fluidly connected to the inlet and an outlet port fluidly connected to the exhaust outlet.

31. A retainer configured to secure the housing to a wall of the structure.

32. A variable output electric ozone generator configured to generate an ozone-rich vicinity within the housing; and.

33. A variable speed electric fan located within the housing, said fan configured to draw air from the internal environment at the inlet and exhaust the air to the external environment at the exhaust outlet after the air passes through the ozone-rich vicinity.

34. Wherein at least one of (the inlet port and the outlet port) is configured for convenient attachment and detachment to a standard-dimension PVC pipe.
